(19) United States
(12) Patent Application Publication
(10) Pub. No.: US 2017/0285006 A1
(43) Pub. Date: Oct. 5, 2017

(54) DEVICE FOR HOLDING AN INDICATOR

(71) Applicant: Nils DANELL, Sodertalje (SE)

(72) Inventor: Nils DANELL, Sodertalje (SE)

(21) Appl. No.: 15/508,231

(22) PCT Filed: Sep. 2, 2015
(86) PCT No.: PCT/SE2015/050922

§ 371 Date: Mar. 2, 2017

(30) Foreign Application Priority Data
Sep. 3, 2014 (SE) 1451023-4

(51) Int. Cl. G01N 33/50; A61F 13/476; A61F 13/471; G01N 33/52

(52) U.S. Cl. CPC G01N 33/5038; G01N 33/526; A61F 13/476; A61F 13/471

(57) ABSTRACT

The present disclosure relates to a device for detection of gaseous amines, originating from disease in the human urogenital system. The device comprising an inner surface 1 for facing the user and outer surface 3 for facing away from the user, wherein said device comprises a removable indicator 5 of gaseous amines. The removable indicator 5 is configured to be arranged in an indicator pocket 6. The device is made of a flexible material of a shape adapted to cover orifices of the human urogenital system but not the anus.

### FIELD OF THE INVENTION

The present invention relates to medical devices and in particular to a device for holding in place an indicator for detection of disease in the urogenital system.

### BACKGROUND OF THE INVENTION

Today, diseases in the urogenital system and urinary tract are discovered late, leading to suffering for individual patients and significant costs for society. A late discovered infection in the urinary tract may necessitate treatment with antibiotics involving medical practitioners and may also lead to serious complications. Early discovery reduces suffering, risk for complications, as well as healthcare costs.

Indicators for detection of gaseous amines produced by microorganisms are known in the art and available from e.g. Siemens Healthcare Diagnostics AB (Upplands Väsby, Sweden).

In the following some patent documents in the general field of personal care products are briefly discussed.

WO2005/061013 discloses an absorbent personal care product comprising an indicator with an amine sensitive dye, placed in the product such that the dye deposit is visible to the unaided eye.

US-2005/0124072 relates to a personal care product with visual indicator of vaginitis.

WO-2011/162657 discloses a male incontinence protector to solve the problem of securely and comfortably retaining the protector in close contact with the body of the wearer.

However, one drawback of the indicator disclosed in WO-2005/061013, and also in US-2005/0124072, is that measurements may be erroneous, due to that gaseous amines from the wearer's anus may influence the measurements, resulting in a detection of a suspected disease in the urogenital system.

Thus, the object of the present invention is to achieve an improved device for detection of disease in the urogenital system giving reliable measurement results, and that is easy to use.

### SUMMARY OF THE INVENTION

The present invention relates to a device according to the independent claim. Preferred embodiments are set forth in the dependent claims.

The present invention provides means for early detection of disease in the urogenital system by providing a device adapted to hold an indicator of amine metabolites produced by microorganisms and affected tissues in place in close proximity to orifices of the human urogenital system, such that gaseous amines from the anus do not influence the measurements. This is essentially achieved by the triangular shape of the device. Particularly the present invention relates to a device provided with an indicator pocket into which an indicator may be removably arranged. The device is specifically advantageous in that it is easy to use, and the resulting measurements are reliable as only gaseous amines from urogenital orifices may reach the active part of the indicator.

### SHORT DESCRIPTION OF THE APPENDED DRAWINGS

FIGS. 1A-1C show various views of one embodiment of the invention.

FIG. 2 shows an embodiment of the invention for use by men.

### DETAILED DESCRIPTION OF PREFERRED EMBODIMENTS OF THE INVENTION

The invention will now be described in detail with references to the appended drawings. Throughout the figures the same or similar items have the same reference signs.

The invention generally relates to a device for detection of gaseous amines originating from disease in the human urogenital system, said device comprising an inner surface for facing the user and and outer surface for facing away from the user, wherein said device comprises an indicator of gaseous amines, and is made of a flexible material of a shape adapted to cover orifices of the human urogenital system but not the anus.

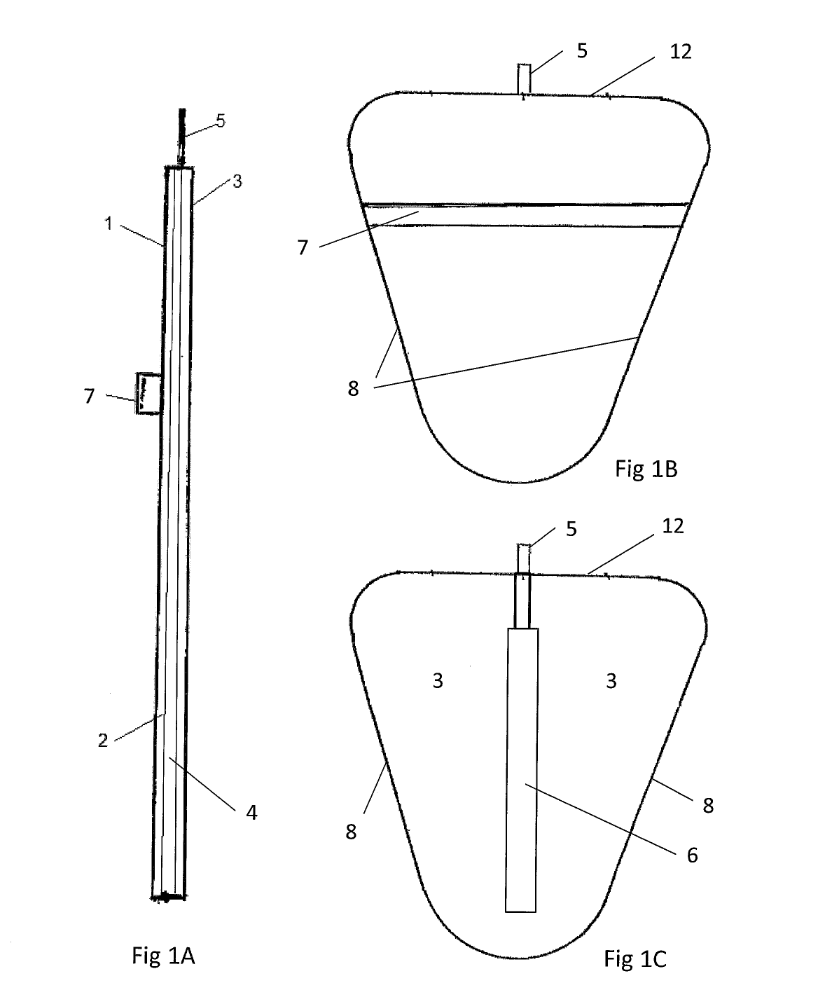

FIG. 1A shows a side view of one embodiment of the device according to the invention. The device is made of a flexible material that may be worn between the genital area and a pair of underpants of a user. Examples of flexible materials are woven and non-woven materials. The device preferably does not contain chlorhexidine or other disinfectants. The device has an outer surface 1 and an inner surface 3. FIG. 1B is a view showing the outer surface side of the device, and FIG. 1C is a view showing the inner surface side of the device.

The device comprises an indicator of gaseous amines 5 that is introduced into the device. According to the invention the indicator 5 is removably attached to the device by providing it in an indicator pocket 6 on the inner surface 3 of the device. The indicator has an elongated shape, being essentially flat, and is made from a soft flexible material having a structural integrity that ensures that it may be easily introduced into the pocket 6. The indicator comprises an active part including an amine sensitive dye, and that the active part of the indicator is positioned such that amine sensitive dye is optimally located to detect amine metabolites produced by microorganisms and affected tissues in place, e.g. in close proximity to orifices of the human urogenital system. In one embodiment the active part is provided close to the distal part of the indicator, being the part that is furthest away from the opening of the indicator pocket when the indicator is correctly inserted into the indicator pocket. The active part is preferably located at both sides of the indicator, which is important in order to secure that a reliable indication may be obtained, i.e. that a part of the active part faces the skin of the patient.

The indicator pocket has a length that corresponds to at least 75% of the length of the indicator. This is important in order for the indicator to be held properly in place during use. The width of the indicator pocket is slightly larger than the width of the indicator. The orientation of the indicator pocket is preferably essentially such that, when the device is arranged to be used, the pocket runs along a vertical symmetry line of the user's body. Thereby the indicator will be arranged in an optimal position in relation to the urogenital orifice of the body.

The device may comprise several layers. It is essential that the layer constituting the inner surface 3 and any further layer between the inner surface 3 and the indicator 5 is permeable to the gaseous amines that are to be detected by the indicator 5. Further layers may include a liquid barrier layer 2 and a moisture absorbing layer 4.

The liquid barrier layer 2 may be any liquid impermeable layer that is sufficiently flexible for inclusion in the device without causing discomfort to the user. Examples of such liquid barrier layers are plastic, e.g. a polypropylene or polyethylene film, or hydrophobic woven and non-woven materials.

The moisture absorbing layer 4 is, if present, preferably made of cellulose such as fluff.

In a preferred embodiment of the invention, the device does not comprise an absorbing layer 4.

In one embodiment of the invention, the device is fitted with a band 7 across the outer surface 1 of the device, wherein said band 7 is arranged to allow a user to hold the device by insertion of a hand between the band and rest of the device, without touching the indicator 5.

In order to prevent the device from sliding towards the anus during use, the shape of the device is generally triangular, as shown in FIGS. 1B and 1C, so that the two edges 8, having equal lengths, of the device abut the groin of the wearer thereby preventing undesired movement. The triangle is an isosceles triangle that has two sides of equal length. More particularly, the device comprises an upper edge 12 being the side of the triangle to be positioned essentially horizontally and upward and being closest to the stomach of the patient.

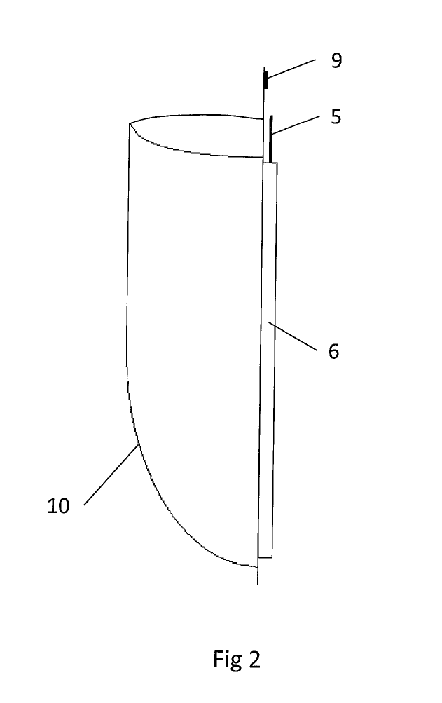

The device may also be provided with means 9, as shown in FIG. 2, for removably attaching the device to an underpant in order to prevent undesired movement. Such means may be i.a. an adhesive patch, velcro, or a hook or catch.

In one embodiment of the invention, the device is adapted for male users. The device then includes a pocket 10 wherein the penis may be put. The pocket 10 is at the side of the device that faces the body, and is preferably made from a gas impermeable material in order to further prevent gas contamination of the indicator 5 by gaseous amines from the anus. However, no gas impermeable material is provided in the part of the pocket 10 close to the indicator pocket 6, in particular close to the lower part of the pocket 10 where the urogenital orifice is located during use.

The indicator 5 may comprise amine sensitive chemichromic dyes that change color in the presence of amines, such as trimethylamine, putrescine and cadaverine commonly produced in infections in the urogenital system. Such indicators are available from inter alia Siemens Healthcare Diagnostics AB (Upplands Väsby, Sweden). One class of such chemichromic dyes that is particularly useful is arylmethane dyes, such as diarylmethanes, triarylmethanes, and the like.

The indicator is preferably provided as a separate item where the active part is protected e.g. by a protective strip that is removed in order to activate the chemichromic dye. The indicator is then inserted into the indicator pocket and kept there during a time period, e.g. a couple of hours. After use the device is removed and the indicator is removed from the indicator pocket. The color of the active part of the indicator is then compared to a color key provided e.g. at a piece of paper, in connection with an application program of a mobile phone, or by another mean, to determine if any amines have been measured.

The present invention is not limited to the above-described preferred embodiments. Various alternatives, modifications and equivalents may be used. Therefore, the above embodiments should not be taken as limiting the scope of the invention, which is defined by the appended claims.

1. Device for detection of gaseous amines originating from disease in the human urogenital system, said device comprising an inner surface (1), having a gas permeable layer, for facing the user and an outer surface (3) for facing away from the user, wherein said device comprises an indicator (5) configured to measure gaseous amines, wherein the indicator is removably placeable in an indicator pocket (6) provided in the gas permeable layer, and that said device is made of a flexible material of a shape adapted to cover orifices of the human urogenital system but not the anus, wherein said shape is generally triangular, and wherein the device is provided with edges (8) configured to abut the groins of the wearer thereby preventing undesired movement.

2. The device according to claim 1, further having a liquid barrier layer.

3. The device according to claim 1, not comprising a moisture absorbing layer.

4. The device according to claim 1, comprising a moisture absorbing layer between the inner surface and the liquid barrier layer.

5. The device according to claim 1, further comprising a band across the outer surface of the device, wherein said band is arranged to allow a user to hold the device by insertion of a hand between the band and rest of the device, without touching the indicator.

6. The device according to claim 1, further comprising means for removably attaching the device to an underpant.

7. The device according to claim 1, further comprising a pocket attached to the inner surface, for holding the penis of the user.

8. The device according to claim 1, wherein the indicator comprises an amine sensitive dye.

9. The device according to claim 1, wherein the indicator (5) has an elongated shape, being essentially flat, and is made from a soft flexible material having a structural integrity that ensures that it may be easily introduced into the indicator pocket (6).

10. The device according to claim 2, not comprising a moisture absorbing layer.

11. The device according to claim 2, comprising a moisture absorbing layer between the inner surface and the liquid barrier layer.

12. The device according to claim 2, further comprising a band across the outer surface of the device, wherein said band is arranged to allow a user to hold the device by insertion of a hand between the band and rest of the device, without touching the indicator.

13. The device according to claim 3, further comprising a band across the outer surface of the device, wherein said band is arranged to allow a user to hold the device by insertion of a hand between the band and rest of the device, without touching the indicator.

14. The device according to claim 4, further comprising a band across the outer surface of the device, wherein said band is arranged to allow a user to hold the device by insertion of a hand between the band and rest of the device, without touching the indicator.

15. The device according to claim 2, further comprising means for removably attaching the device to an underpant.

16. The device according to claim 3, further comprising means for removably attaching the device to an underpant.

17. The device according to claim 4, further comprising means for removably attaching the device to an underpant.

18. The device according to claim 5, further comprising means for removably attaching the device to an underpant.

19. The device according to claim 2, further comprising a pocket attached to the inner surface, for holding the penis of the user.

20. The device according to claim 3, further comprising a pocket attached to the inner surface, for holding the penis of the user.
